(19) United States
(12) Patent Application Publication
(10) Pub. No.: US 2018/0116957 A1
(43) Pub. Date: May 3, 2018

(54) METHOD OF PRODUCING OF PHARMACOLOGICALLY ACTIVE LIPOSOMAL COMPOSITION CONTAINING CYTOCHROME C, AND LIPOSOMAL COMPOSITION OBTAINED BY THIS METHOD

(71) Applicant: TOVARISTVO Z OBMEZHENOU VIDPOVIDALNISTU "NANOMEDTRAST", Kyiv (UA)

(72) Inventors: Ganna S. GRYGOR'IEVA, Kyiv (UA); Oleksii H. KATSAI, Kharkiv (UA); Yurii M. KRASNOPOLSKYI, Kharkiv (UA); Vitalii V. PROKHOROV, Kharkiv (UA); Oleksandr S. KHROMOV, Kyiv (UA); Nataliya V. PASYECHNIKOVA, Kyiv (UA); Natalia V. DOBRELIA, Kyiv (UA)

(21) Appl. No.: 15/793,184

(22) Filed: Oct. 25, 2017

(30) Foreign Application Priority Data
Oct. 27, 2016 (UA) A201610776

(51) Int. Cl. A61K 9/127; A61K 38/41; A61K 47/54

(52) U.S. Cl. CPC A61K 9/127; A61K 47/544; A61K 38/415

(57) ABSTRACT

This invention relates to pharmaceutics and a method of producing of liposomal composition containing cytochrome c and the pharmacologically active liposomal composition obtained by this method, which can be used as a means of polyfunctional pharmacotherapy, especially for ophthalmology, hematology and cardiology.

A method of producing of liposomal composition containing cytochrome c, the method includes preparing mixture of solutions of lipids in an organic solvents, drying in vacuum the mixture and emulsifying it in aqueous medium containing cytochrome c, adding of cryoprotectant, homogenization of the emulsion, filtration and freeze-drying, and according to the invention the lipids are egg or soybean phosphatidylcholine with one or two of lipids from the group consisting of dipalmitoylphosphatidylglycerol, dipalmitoylphosphatidylcholine, distearoylphosphatidylcholine, diphosphatidylglycerol, phosphatidylglycerol, phosphatidylinositol or dioleoyloxypropy trimethylammonium, whereas the ratio of phosphatidylcholine:to other lipids is 0.3-2.0:1, thereby to prepare the mixture of solutions of lipids, phosphatidylcholine is dissolved in ethyl alcohol, and other lipids—in chloroform and a volume ratio in a mixture of solutions of ethyl alcohol:chloroform is 1:1.5-2.5, emulsification is performed at a weight ratio of cytochrome c to lipids of 1:11.4-18.5 adding a cryoprotectant solution to the aqueous medium, and the cryoprotectant is selected from lactose, trehalose, sucrose oligosaccharides, wherein the said solution contains 60-80% of total cryoprotectant, and homogenization with step-by-step increasing pressure from 300 to 800 atm, after its completion a solution of selected cryoprotectant is added to emulsion, wherein solution contains 40-20% of total cryoprotectant, and the weight ratio of lipids mixture to cryoprotectant is 1:5.5-7.2.

It is claimed a liposomal composition containing cytochrome c, lipids and cryoprotectant, which is performed according to the method above, and the composition includes egg or soybean phosphatidylcholine in mixture with one or two of lipids from the group consisting of dipalmitoylphosphatidylglycerol, dipalmitoylphosphatidylcholine, distearoylphosphatidylcholine, diphosphatidylglycerol, phosphatidylglycerol, phosphatidylinositol or dioleoyloxypropy trimethylammonium, and cryoprotectant is selected from lactose, trehalose, sucrose oligosaccharides, wherein the weight ratio of cytochrome c:phosphatidylcholine:other lipids:cryoprotectant is 1:2.9-8.6:4.3-15.7:78.6-102.8 and percentage content ratio is (0.81-1.06)%:(3.03-8.26)%:(4.13-9.88)%:(78.67-83.30)% respectively.

This invention relates to pharmaceutics and a method of producing of liposomal composition containing cytochrome c and the pharmacologically active liposomal composition obtained by this method, which can be used as a means of polyfunctional pharmacotherapy, especially for ophthalmology, hematology and cardiology.

Cytochrome c is a small protein (m. m. 12 kDa) that is present in practically all aerobic organisms and is involved in redox processes of cellular respiration performing an enzyme function of the respiratory chain in mitochondria. An enzyme prosthetic group contains iron which reversibly goes from an oxidation state to reduced state. Cytochrome c can accelerate metabolic processes in tissues, improve oxygen utilization and reduce effects of pathological hypoxic and toxic impact [1].

These factors determining physiological multi-vector functions of cytochrome c became a basis of its use in pharmacotherapy. There is number of drugs based on cytochrome c active substance from different manufacturers in a dosage form for parenteral administration and in a form of drops (eye drops): “Cytochrom C” of “Farmstandart-Biolik” in Ukraine; “Cytochrom C”, of “Sumson-med” in Russian Federation, “CytoMak” of “Heinrich Mark” in Germany, “Cytochrom C” of “Defeier Pharm.” in China, “Oftan-Katachrom” of “Santen Oy” in Finland, “Vitafacol” of “Ciba Vision Ophtalmics” in France and etc. [2] In addition to the universality of enzyme redox properties an effect of these drugs is directed to the restoration of level of endogenous cytochrome c, which is decreased in the targeted organs due to injuries (in particular in lens during cataract) [3].

However, the clinical use of cytochrome c is accompanied by a number of side effects, among which the most common are allergic reactions, hypotension, dizziness (injection solution), burning eyes and contact dermatitis (eye drops). Manifestation of these undesirable reactions is greatly enhanced due to long time medicine course of use foreseen by the guidelines for the clinical application of all known cytochrome c drugs: up to 25 days for injectable forms and up to 6 months for the eye drops which is caused by peculiarities of bioavailability of cytochrome c water-soluble substance (its ineffective penetration to different eye structure) and its rapid excretion from an organism [2, 4].

The advisability of extension of the pharmacological use of cytochrome c essential properties along with optimization of the benefit/risk ratio and increasing of targeting makes creating of new dosage forms of cytochrome c drugs and development of methods to obtain such drugs an actual task.

The current requirements for such methods and products fully correspond to obtainment of liposome based drugs, and it is associated with a priori physiological advantages of liposomes [5]: natural biocompatibility of the lipid matrix of these nanoparticles with the organism; programmability of transport; selective depositing in tissues which are in a state of hypoxia; non-antigenicity and absence of systemic toxicity or significant side effects.

The liposomal organization of active pharmaceutical substances of various classes [5, 6] is an innovative direction of modern pharmacy, as reflected in regulatory documents of the world regulatory bodies (FDA, 2014: Recent requirements for innovated liposomal drugs and liposomal nanosimilars; EMA, 2015: Guidelines and regulatory aspects of liposomal drugs; State Pharmacopoeia of Ukraine, 2015: Liposomal drugs N:).

These days in the world according to the different clinical statements more than 60 liposomal drugs are licensed (Doxil, Lipodox, Daunoxel, Depocyt, Myocet—for oncology, Ambisom—for antifungal therapy, Lioliv—for gastroenterology, Lipin—for pulmonology, nephrology and obstetrics, Lipoflavon—for cardiology, oncology, Vizudin, Lipoflavon, Lipoferon—for ophthalmology, etc.).

The effectiveness of liposomal drugs use in different segments of the clinic has allowed to predict positive effects of method development for inclusion of cytochrome c substance to liposomes in order to obtain a product that combines multifunctionality of pharmacotherapycal influence and harmlessness.

There are published results of scientific research findings on liposomal systems with cytochrome c [7-9]. The main purpose of these scientific findings is to determine the variability of liposomes lipid composition and parameters of non-validated experimental methods for its formation (for example, thin-layer evaporation, extruding with force through glass pores of different densities, etc.), as well as an assessment of its potential pharmacological effect. In these researches, size of the resulting lipid vesicles varies from 100 nm to 10.000 nm, and the encapsulation of the cytochrome c substance does not exceed 50-60%.

It is described methods for obtaining of natural lipids based liposome compositions with cytochrome c [10-13].

According to [10], the preparation of cytochrome c liposomal form is performed by obtaining a film of a mixture of negatively charged lipids from organic solution, followed by suspension of cytochrome c aqueous solution in a saline solution, and by extruding the suspension on the extruder. Since the ratio of lipids in the mixture is not identified, and exceptionally evaluative measurements of absorbance spectrum in a range from 400 nm to 700 nm was used in order to specify a formation of liposomes, their size and cytochrome c content (i.e. “turbidity spectrum”), therefore the liposome nature, particles size and introduction of the cytochrome c to a targeted product can not be considered as confirmed and reliable.

The method known from [11] involves dissolution of soybean phosphatidylcholine, a complex of anionic soybean phospholipids, cholesterol and vitamin E in chloroform at a weight ratio of 55:25:10:1 respectively, mixing these solutions with subsequent drying and emulsification into cytochrome c aqueous solution, emulsion dispersing by ultrasound, and sterile filtration. The ratio of total lipids to cytochrome c in the product is 8:1. This method provides inclusion of only 55-60% of the introduced cytochrome c to liposomes (i.e. obtained composition comprises almost a half of the “free” form of substance). Moreover, essential disadvantage of this method is use of an ultrasound for dispersion of emulsion that adversely affects the quality of a liposomal product which has a big degree of oxidation (a peroxide number of 0.1-0.2), and also it makes impossible to produce a standardized composition under industrial conditions.

It is known a method for producing of a liposomal composition with cytochrome c, consisting of natural phospholipids, cholesterol, sitosterol, polyethylene glycol 400, glutathione additives and ascorbic acid [12], the methods include drying of phospholipids and cholesterol mixture with following emulsification of the film in the cytochrome c solution and of another water-soluble constituents in phosphate buffer, dispersion and sterile filtration of the obtained liposomal emulsion. Nevertheless, this method and product of its implementation has essentials disadvantages: unreasonably large amount of cholesterol (20-40% wt) that increases stiffness of liposomes membrane and extends duration of the sterile filtration stage; heterogeneity of liposomes size in a range from 50 nm to 120 nm; absence of confirmation of the encapsulation of cytochrome c to liposomes, i.e. the received liposomal composition is not standardized.

There is a known method for obtaining a liposomal form of cytochrome c [13] containing cytochrome c and a mixture of two lipids, dipalmitoylphosphatidylglycerol and phosphatidylcholine. The mentioned method and a product of its implementation is selected as a prototype for the claimed subject matter—a method of producing of liposomal composition containing cytochrome c, and liposomal composition obtained by this method, i.e. as the closest analogue according to the general features, namely with accordance to: essence and sequence of the basic method operations, the liposomal organization of the composition containing cytochrome c, and the nature of some components in the composition.

The said method of producing a liposomal form of cytochrome c [13] involves drying in vacuum the mixture of dipalmitoylphosphatidylglycerol and phosphatidylcholine given at weight ratio of 1:1.2-4.0 from solution of chloroform and ethyl alcohol mixture, emulsifying the mixture in aqueous solution containing the cytochrome c, wherein weight ration of cytochrome c to lipids mixture is 1:29.33-66.66, homogenization of the emulsion at a pressure of 600-1200 atm, in a process of which a cryoprotectant lactose is added at a weight ratio of a mixture of lipids and lactose 1:3.0-5.5, and sterile filtration followed by freeze-drying.

However, according to the prototype, the method operations is performed under certain conditions that can negatively affect the processing of method itself and quality of the targeted product, and the obtained liposomal composition does not provide optimal foreseen pharmacotherapeutic effect.

Firstly, on implementation of the prototype, dissolution of both lipids, phosphatidylcholine and dipalmitoylphosphatidylglycerol, is carried out in a mixture of chloroform and ethyl alcohol (with a ratio of 4:1) extending the dissolution time, and therefore can contribute to the oxidation of substances.

Secondly, the prototype involves simultaneous injection of all used amount of cryoprotectant lactose during dispersion of the emulsion, as well as conducting the entire process of dispersion at fixed pressure. As a result, these factors lead to an extension of process duration, and in the future may negatively affect the freeze-drying operation and homogeneity of liposomes size. The second factor is particularly important in view of the stated ophthalmic purpose, according to the prototype, of the targeted product. Thus, with accordance to the prototype, obtained liposomal composition with cytochrome c contains up to 10% so called “small” liposomes having size up to 75 nm, predictably worsening the pharmacological effect when using the product in the form of eye drops [6].

Thirdly, for the implementation of the prototype method, it is proposed a ratio of 1:29.33-66.66 of cytochrome c to lipids mixture that is a contribution of an active substance is much less than of lipid component, i.e. liposomes matrix. Furthermore, mixture of lipids, despite the general definition, is represented only by two specified lipids, and the content of neutral phosphatidylcholine is bigger (up to 4 times) than content of anionic dipalmitoylphosphatidylglycerol. Considering that a charge of the lipid affects the pharmacological activity of the liposomal product (negative charge can extend the time of its retaining in the targeted organ), the mentioned factors do not allow to recognize the applied prototype ratio of components to be optimal for producing of composition with the desired efficacy.

It should be noted that in the wording of the claims according to the prototype, certain provisions do not go in line with the corresponding statements in examples illustrating these claims (Examples No. 1-4). Thus, according to the claims the ratio of cytochrome c to lipids should be 1:29.33-66.66, but with accordance to the examples it is actually 1:16.3-37.0. The prototype claims involve pressure changes, at which homogenization of the emulsion is carried out, in the range of 600-1200 atm, although in all examples this process is performed only at a fixed pressure of 900 atm. In the prototype claims statement about producing of “liposomal drug for ophthalmology” is declarative, since it is not supported by any evidence in favor of the pharmacological activity of the composition, including its predicted ophthalmic specificity.

These facts reduce the effectiveness and reliability of the prototype method in relation to the process of its implementation, as well as the quality and stability of the targeted product as a pharmacologically active liposomal form of cytochrome c.

The present invention claims development of a method of producing of a liposomal composition containing cytochrome c with optimized parameters of operations, the method provides improved quality and stability of the targeted product, and thereby obtaining a liposomal composition containing cytochrome c with an optimal components and pharmacological properties that is adequate for use as a multifunctional pharmacotherapy drug.

The object matter is solved through a method of producing of liposomal composition containing cytochrome c, the method includes preparing a mixture of solutions of lipids in an organic solvents, drying in vacuum the mixture and emulsifying it in aqueous medium containing cytochrome c, adding of cryoprotectant, homogenization of the emulsion, filtration and freeze-drying, and according to the invention the lipids are egg or soybean phosphatidylcholine with one or two of lipids from the group consisting of dipalmitoylphosphatidylglycerol, dipalmitoylphosphatidylcholine, distearoylphosphatidylcholine, diphosphatidylglycerol, phosphatidylglycerol, phosphatidylinositol or dioleoyloxypropy trimethylammonium, whereas the ratio of phosphatidylcholine to other lipids is 0.3-2.0:1, thereby to prepare the mixture of solutions of lipids, phosphatidylcholine is dissolved in ethyl alcohol, and other lipids—in chloroform and a volume ratio in a mixture of solutions of ethyl alcohol:chloroform is 1:1.5-2.5, emulsification is performed at a weight ratio of cytochrome c to lipids of 1:11.4-18.5 adding a cryoprotectant solution to the aqueous medium, and the cryoprotectant is selected from lactose, trehalose, sucrose oligosaccharides, wherein the said solution contains 60-80% of total cryoprotectant, and homogenization with step-by-step increasing pressure from 300 to 800 atm, after its completion a solution of selected cryoprotectant is added to emulsion, wherein solution contains 40-20% of total cryoprotectant, and the weight ratio of lipids mixture to cryoprotectant is 1:5.5-7.2.

The object matter is also solved by result of the implementation of the new said method, which is an identified liposomal composition containing cytochrome c, lipids and cryoprotectant, and according to the invention the composition includes egg or soybean phosphatidylcholine with one or two of lipids from the group consisting of dipalmitoylphosphatidylglycerol, dipalmitoylphosphatidylcholine, distearoylphosphatidylcholine, diphosphatidylglycerol, phosphatidylglycerol, phosphatidylinositol or dioleoyloxypropy trimethylammonium, and cryoprotectant is selected from from lactose, trehalose, sucrose oligosaccharides, wherein the weight ratio of cytochrome c:phosphatidylcholine:other lipids:cryoprotectant is 1:2.9-8.6:4.3-15.7:78.6-102.8 and percentage ratio is (0.81-1.06)%:(3.03-8.26)%:(4.13-9.88)%:(78.67-83.30)% respectively.

In compliance to the invention, a differently directed pharmacological activity is established for the produced composition: in ophthalmology (anti-cataract effect), hematology (restoration of blood coagulation system in acute massive blood loss) and cardiology (anti-hypoxic effect), as well as antitoxic activity.

The following examples illustrate the possibility of implementation of the claimed method, and according to it producing of the targeted product-composition, and for comparison—producing of an example by the prototype method.

For convenience, conventional names of lipids are indicated, which also are used in the disclosure of the claimed method and composition as well as for the prototype.

| Lipid rational name | Conventional name |
|---|---|
| Phosphatidylcholine | PC |
| Egg phosphatidylcholine | (e)PC |
| Soybean phosphatidylcholine | (s)PC |
| Dipalmitoylphosphatidylglycerol | DPPG |
| Dipalmitoylphosphatidylcholine | DPPC |
| Distearoylphosphatidylcholine | DSPC |
| Diphosphatidylglycerol | DPG |
| Phosphatidylglycerol | PG |
| Phosphatidylinositol | PI |
| Dioleoyloxypropytrimethylammonium | DOTA |

### EXAMPLE 1

Claimed invention. The accurately weighed 6.0 g of (e)PC (in terms of 100% substance [for example, 14]) is dissolved in 100 ml of ethyl alcohol while stirring. The accurately weighed 5.0 g of DPPG (in terms of 100% substance [14]) is dissolved in 150 ml of chloroform while stirring, and after combining the resulted solutions with the (e)PC alcoholic solution (volumetric ratio of ethyl alcohol to chloroform is 1:1.5). A mixture of lipid solutions is filtered through a membrane with a pore diameter of 0.22 μm, transferred to a rotary evaporator, and the solvents are removed by drying in a vacuum at a temperature of 40-45° C. until a thin film is obtained. Upon completion of the drying process, an inert gas is passed to the evaporator flask within 25-45 minutes. The accurately weighed 0.70 g of cytochrome c (in terms of 100% substance [for example, 15-16]) is dissolved in 80 ml of sterile phosphate buffered solution of pH 6.7-7.1, and filtered through a membrane with a pore diameter of 0.22 μm. The accurately weighed 60.0 g of lactose (pharmacopeial milk sugar) in terms of 100% substance at a temperature of 50-60° C. is dissolved, while stirring, in 250 ml of sterile phosphate buffered solution of pH 67-7,1 and filtered through a membrane with a pore diameter of 0.22 μm.

After drying, the resulting lipid film is quantitatively removed from the walls of the evaporator's flask using a mixture of 620 ml of sterile phosphate buffered solution of pH 6.7-7.1, 80 ml of cytochrome solution containing 0.7 g of cytochrome c, and 240 ml of lactose solution in a phosphate buffer containing 48.0 g of lactose, while stirring for 60 minutes at 100-120 rpm (for example, IKA, Germany) until a homogeneous emulsion is obtained.

The emulsion is transferred to a reactor of high pressure homogenizer (for example, M 110R Microfluidizer Processor, Microfluidics) and is dispersed at a temperature of 38-45° C. with a step-by-step increasing pressure from 300 atm to 800 atm for 1-3 cycles. The size of the emulsion particles (for example, Malvern Zetasizer Nano S) at the end of the dispersion process does not exceed 160 nm.

After completion of homogenization, 60 ml of a sterile lactose solution in a buffer solution of pH 6,7-7,1 containing 12.0 g of lactose is added to the emulsion and is mixed together. The volume of the emulsion is 1000 ml. The resulting emulsion is filtered through a membrane with a pore diameter of 0.22 μm, and it is then a subject for sterile filtration, after that it is dosed in an aseptic manner in glass vials.

The vials with the emulsion are subjected to intensive freezing and freeze-drying (e.g., Martin Christ-2-6-D, USA). After drying, the vials with the lyophilized product are sealed in an inert gas atmosphere under aseptic conditions.

In the Examples 2-12, the claimed method is carried out in accordance with the Example 1. Changes in the parameters of the process implementation are reflected in Tables No. 1-3.

The targeted product is a light amorphous mass of yellow color with a characteristic smell.

### EXAMPLE 13

Prototype according to [13]. PC in the amount of 3.6 g and DPPG in the amount of 1.0 g (in terms of 100% substance) is dissolved in 150 ml of a mixture of chloroform and ethyl alcohol (ratio of 4:1), and stirred. A lipid solution is filtered through a membrane with a pore diameter of 0.22 μm, transferred to a rotary evaporator and evaporated at a temperature of 41-45° C. until a thin film is obtained. The film is treated with nitrogen gas for 15-20 min. The accurately weighed 0.135 g of cytochrome c (in terms of 100% substance) is dissolved in 135 ml of sterile phosphate buffered solution of pH 6.5-6.8 (cytochrome c concentration is 1 mg/ml) and filtered through a membrane with a pore diameter of 0.22 μm. The accurately weighed 14.0 g of lactose, in terms of 100% substance, is dissolved in 35 ml of sterile phosphate buffered solution of pH 6.5-6.8 (lactose concentration 400 mg/ml) and filtered through a membrane with a pore diameter of 0.22 μm.

The resulting lipid film is quantitatively removed from the walls of the evaporator flask with a mixture of 135 ml of cytochrome c solution (concentration of 1 mg/ml) and 30 ml of sterile phosphate buffered solution of pH 6.5-6.8. The content of the flask is stirred for 2 hours until a homogeneous emulsion is obtained.

The emulsion is transferred to a high pressure homogenizer, and dispersed at a temperature of 38-44° C. under pressure of 900 atm to obtain a particles size that does not exceed 200 nm. After achieving this particle size, 35 ml of lactose solution (containing 14 g of lactose) is added to the emulsion. The dispersion is continued until particles size is not more than 130-150 nm.

The resulting emulsion is filtered through a cascade of filters, wherein terminal filter has pores size of 0.22 μm, and then sterile filtered, bottled into glass vials, freeze-dried and sealed under nitrogen atmosphere.

Based on the results of implementation of the prototype method, the product is in the form of a light amorphous mass of yellow color with a characteristic smell.

When identifying and establishing the quality parameters of a liposomal compositions obtained according to the proposed and prototype method, the compositions were used per se and in the form of an emulsion, reconstituted by adding in a vial with a lyophilized product of a sterile isotonic solution corresponding to the form of their potential pharmacotherapeutic application.

The efficacy of the claimed method with respect to the pharmaceutical quality of the produced liposomal composition is confirmed by the results of qualitative and quantitative identification of cytochrome c and lipid components (PC and other lipids), and the liposomal status of the targeted product is confirmed using a number of independent physical and chemical methods, namely:
In spectrophotometric method, by the characteristic absorption spectrum in a range of 400-600 nm and an optical density of solution at wavelengths (407±2) nm and (490±2) nm, the solution is obtained after holding the targeted product in water at a temperature of 35° C. for 30 min, in comparison with such a solution of the standard sample of cytochrome c (identification and quantitative determination of cytochrome c respectively);
In sodium dodecyl sulfate polyacrylamide gel electrophoresis (SDS-PAGE), by the electrophoregram of aqueous solution of the targeted product in comparison with such a solution of the standard sample of cytochrome c (identification of cytochrome c respectively);
Spectrophotometric method by the characteristic absorption spectrum of solution in a range of 450-650 nm with maximum at wavelength of 522-526 nm and 559-561 nm, the solution is obtained after holding the targeted product in water at a temperature of 35° C. for 30 min, followed by passing of nitrogen(II) oxide until appearance of a bright pink color (identification of the iron-containing prosthetic group of cytochrome c);
In thin-layer chromatography, by a chromatogram of the targeted product solution in a mixture of chloroform, methanol and water (volumetric ratio 73:23:3), the chromatogram contains spots of PC and other lipids at the level of the main spots on the chromatograms of solutions of standard samples of PC and other corresponding lipids (identification of PC and other lipids);
In liquid chromatography technic with evaporative light scattering detector (ELSD), by a chromatogram of a solution of the targeted product in a mixture of chloroform, methanol and water (volumetric ratio 73:23:3) using for calibration and comparison a solution of standard samples of PC and corresponding lipids (quantitative determination of PC and other lipids);
In liquid chromatography technic with spectrophotometric detection at a wavelength of 409 nm, by a chromatogram of solution obtained after holding the targeted product in a water at room temperature for 30 min, compared to the standard sample of the “free” cytochrome c (level of encapsulation of cytochrome c to liposomes);
By measuring the size of the particles in emulsion of liposomal product by the method of dynamic light scattering (DLS);
By index of oxidation of the targeted product lipid fraction (stability of liposomes);
By parameters of formation time, separation stability and dispersed composition of the emulsion reconstituted from the freeze-dried targeted product (functional stability and distribution of liposomes by size);
By pH and osmolality of the emulsion (determination of compliance with the requirements of functional use of parenteral and ophthalmic drugs).

According to the results of physical and chemical analysis (Table 4), the claimed method provides the pharmaceutical quality and stability of the targeted product as a liposomal composition containing cytochrome c, namely:
With accordance to the methods of spectrophotometry, thin-layer chromatography and liquid chromatography, the targeted product keeps the native composition and nature of cytochrome c, PC and others selected lipids, which coincides with the corresponding standards of cytochrome c, PC and the corresponding lipids;
Cytochrome c is quantitatively incorporated into liposomes with a level of encapsulation of more than 90%;
The targeted product is characterized by quick formation of the emulsion from lyophilisate and emulsion separation stability;
An emulsion of the targeted product has the stable liposome average size of 147±3 nm accompanying by a practical monodispersity of their distribution by size (90-100%), including prolonged storage, and low oxidation index;
Emulsion pH value is stable and it corresponds to the physiological norms for this factor in vivo;
The osmolality index corresponds to pharmacopoeial requirements for ophthalmic drugs.

It should be noted that optimal pharmaceutical quality and stability is inherent to the targeted product produced by Examples 1-8, and the implementation of the claimed method, with excellent parameters (Examples 9-12), and of the prototype method (Example 13) causes a negative distortion of these parameters (Table 4), namely:
decreasing of encapsulation level of cytochrome c into liposomes (by 2.8-7.0% in the examples 9-12 and 6.8-11.0% in the prototype);
significant heterogeneity of liposomes dispersion by size (60-25-15%, 80-10-10% and 75-25% in examples 9,11 and 12 respectively, and 67-33% in the prototype), which increases during storage of the product, having tendency to increase the size of liposomes;
relative index rise of oxidation of the liposomal product;
increasing of time of emulsion formation from the freeze-dried product (in 1.5-2 times) simultaneously with decreasing of its separation stability (in 1.3 times);
relative increasing of osmolality of the emulsion (for 10-40 mOsm/g until the upper limit of the pharmacopoeial norm of 380 mOsm/g for ophthalmic drugs).

The said important advantages of the pharmaceutical quality and stability of the composition produced by Examples 1-8 are the consequence and are coupled with optimized performance of the claimed method (Table 3) compared to the process parameters provided by Examples 9-12 and the prototype (Example 13), in particular:
different dissolution in various solvents—PC (in ethanol) and other lipids (in chloroform), followed by their merge at final ratio of ethyl alcohol:chloroform 1:1.5-2.5 (comp.:according to the prototype, analogical use of mixture of ethanol:chloroform 1:4 for dissolving in the Examples 9 and 12 resulted in deterioration of the pharmaceutical quality of the product);
reduction of time in 1.5-2.6 for stirring after emulsification;
dispersion within three successive cycles at a pressure in the range of 300-800 atm;
two-stage introduction of cryoprotectant: at emulsification (60-80%) and after dispersion (40-20%) (compar.: introduction of all or part of cryoprotectant amount at the dispersion process in the Examples 9 and 11 resulted in deterioration of the pharmaceutical quality of the product);
no filtration on cascade filters, therefore the total time of filtration is reduced in 2.1-2.9 times.

Thus, the claimed method which is performed according to the Examples 1-8, provides producing of a liposomal composition containing cytochrome c having competitive high pharmaceutical quality and stability, along with certain technological advantages of this method. It is important that this conclusion is true for the implementation when using all of the lipids and cryoprotectants of various nature selected in Table 1, which considerably extend the limits of method use.

All characteristics of the pharmaceutical quality of the product of claimed method are established according to validated methods. The composition content is calculated basing upon reliable data on the content of cytochrome c, PC and other lipids in the product which was obtained by the claimed method and the prototype method, by weight ratio and percentage content of the components (Table 5). While calculating the percentage composition content (for 100%), an experimentally determined index of weight loss during drying was taken into account.

In accordance with the object of the invention, the quality of the produced liposomal composition of cytochrome c is evaluated by pharmacological activity factors in preclinical studies at various pathological conditions and methods of administration.

The selected specificity of the studies of the produced composition corresponds to the object of the invention, with reference to the proof of the production of the targeted product with polyfunctional pharmacological activity.

A comparison of the specific pharmacological effect of a liposomal composition containing cytochrome c obtained by the claimed method and prototype method was carried out in experimental models of the following pathologies:

1) a model of irradiation cataract caused by chronic general irradiation of animals (rabbits) with polychromatic light in a range of 350-1150 nm for 26 weeks, which according to clinical features corresponds to hard nuclear cataract in humans [17].

The composition in question was administered in a form of drop emulsion (0.675 mg/ml of cytochrome c). Instillations (three times a day) were began on the 21st week after the created cataract model, they were continued for 6 weeks.

As an analogue of functional activity in the evaluation of the pharmacological effect of the liposomal composition, the cytochrome c drug “Oftan-Katachrom” (“Santen Oy”, Finland) was used. An aqueous solution containing nicotinamide, adenosine and benzalkonium chloride (20, 2 and 0.20 mg/ml respectively), which is similar to the solvent of the “Otan-Katachrom”, was used to correctly compare the effects of the products to create the emulsion of the liposomal composition. Animals of the control group received instillations of the same solvent in appropriate doses.

Evaluation of the pharmacological effect of a liposomal composition containing cytochrome c, which is obtained by the claimed and prototype method in the cataract model, was performed according to standards for assessing the state of the eye lens (5 stages of cataract: no changes—stage 0 and intense cataract lesion—stage 5) [18]. The biochemical parameters were also determined (enzymes and lipid peroxidation products (LOPs)) in aqueous humour in chamber and lens.

2) a model of hemostatic system damage due to acute massive blood loss (MBL) in volume of 30% of circulating blood of animals (sexually mature white rats), which leads to disseminated intravascular coagulation of blood with metabolic acidosis [19, 20].

The composition in question was administered 30 minutes after MBL, intravenously, in the form of an emulsion in a saline solution (1 mg/kg cytochrome c) in volume corresponding to the volume of lost blood (isovolumic replenishment).

The evaluation of the pharmacological effect of the liposomal composition containing cytochrome c, which is obtained by the claimed and the prototype method in the MBL model, was performed according to the parameters of the coagulation system, acid—base homeostasis balance state and gas exchange of arterial blood of animals.

3) models of normobaric, gemic and histotoxic (tissue) hypoxia, which are used to assess the anti-hypoxic effect of potential drugs [21].

Normobaric hypoxia was caused by placing experimental animals in a hermetic chamber with a closed volume of living space of 0.5 l. Gemic hypoxia was caused by the introduction of methemoglobin-forming substance to the animals, i.e. sodium nitrite at a dose of 200 mg/kg. Tissue hypoxia was modeled by the introduction of nitroprusside sodium according to the following scheme: 1st-4th day—1 mg/kg (once a day), 5th day—25 mg/kg. In all models of hypoxia in experiments, adult white mice of both sexes are used.

The products in question were administered as intraperitoneal injection at a dose of 2 mg/kg (basing on cytochrome c content) according to the following scheme: 1st-4th day—once a day, 5th day—in 30 min before hipoxia modelling.

The criterion for estimating the anti-hypoxic effect of a liposomal composition containing cytochrome c, which is obtained by the claimed and prototype method, was the lifetime of the experimental animals.

4) a model of narcotic intoxication [21] caused by administration of thiopental sodium in a dose of 45 mg/kg to adult white rats of both sexes.

The products in question were administered as intraperitoneal injections in 30 min before the introduction of xenobiotic at a dose of 1 mg/kg (basing on cytochrome c content).

The criterion for estimating the antitoxic effect of a liposomal composition containing cytochrome c, which is obtained by the claimed and prototype method, was the time of narcotic sleep.

For a correct assessment of the pharmacological effect in the models of MBL, hypoxia and narcotic intoxication cytochrome c solution in saline solution was used as an analogue of the liposomal composition with respect to the functional activity (for example, Farmasino Pharm. Co., Ltd, China) in equimolar amounts based on cytochrome c content. To animals of the control group saline solution in appropriate doses were injected.

According to the current requirements for the pharmacological products considered as potential medicinal products, as well as to confirm the correctness of the dose regimen and method of administration, the harmlessness of the liposomal composition containing cytochrome c, which is obtained by the claimed method and the prototype method, with respect to the provided methods of its administration (injections and eye drops), was assessed.

The applied products in the form of an emulsion in a saline solution with single intravenous administration and repeated intraperitoneal injections (14 days) did not cause death of experimental animals (white rats), local irritation, negative influence on body weight and weight coefficients of internal organs, changes in blood formulas and basic biochemical parameters of blood serum.

While studding acute and chronic (28 days) ophthalmological toxicity of the liposomal composition containing cytochrome C, and the composition is in the form of drops in the saline solution, no negative manifestations were found regarding the structures of the anterior part of the eye and the optic disc, as well as the locally irritating and allergic effect (on rabbits).

Thus, according to the characteristics of acute and chronic toxicity, including ophthalmological harmlessness, that do not differ for the liposomal composition containing cytochrome c, which is obtained by the claimed method and prototype method, they should be attributed to virtually harmlessness means, which makes possible the potential pharmacotherapeutic use.

In order to comply with the bioethical regulations governing the rules of humane treatment of animals and minimizing animals number in preclinical studies [22], in highly invasive experimental models (acute massive blood loss, cataract with lens removal), separate indicative examples of a liposomal composition containing cytochrome c obtained by the claimed method, are used. For optimal balance between adherence to bioethical regulations and the reliability of estimation of pharmacological activity in models of different pathologies, examples of compositions have been selected on a cross-sectional basis, and in the models of hypoxia (less invasive), all samples of the composition with the highest pharmaceutical quality (Examples 1-8) were analyzed, and for comparison—also examples 9 and 10. To be noted, the pharmacological effect of the product produced by the prototype method was analyzed in all experimental models (Example 13).

Tables 6-8 show the results of establishing the pharmacological effect of a liposomal composition containing cytochrome c, which is produced by the claimed method and prototype method. In all models of pathologies, the products have pharmacological activity, but the very implementation of the claimed method provides producing of a composition with a high polyfunctional pharmacological effect.

In the model of irradiation without treatment (negative control) in animals, severe 3 and 4 stages of damages caused by cataract (50% of eyes with cataract of each stage correspondingly) are achieved, which is accompanied by pathological changes in the biochemical parameters of the lens (accumulation in 1.7 times of products of LOP and imbalance of the enzymes activity in 1.4-1.7 times). At the same time the claimed liposomal composition of cytochrome c shows high effect against cataract (Table 6):
prevents cataract development reducing the lesion of the lens to the initial stages 1 and 2 (12.5-25% and 87.5-75% of eyes amount, correspondingly), in the absence of manifestations of cataract with severe irreversible stages 3 and 4;
virtually normalizes an enzymes activity and the content of LPO products in the lens (89-95% of the norm).

In the MBL model, lesions of the hemostasis system are manifested in reducing of parameters of blood coagulation for 15-30% and distortion of the acid—base homeostasis state and gas exchange in blood (in particular, a decreasing of pH for 0.243 units to non-physiological values and reducing for 10% to critical range of oxygen saturation of blood).

The liposomal composition of cytochrome c, which is produced by the claimed method, has the ability to restore the major components of the hemostasis system which were affected by the MBL, causing (Table 7):
restoration of parameters of the functional state of the blood coagulation system (up to 88-99% of normal) with a clear tendency to support coagulation hemostasis (thrombocytopenia—the level of APTT, PT, TT and fibrinolysis—the level of fibrinogen);
practical elimination of decompensated metabolic acidosis according to the acid-base homeostasis state (increasing of pH and BB to physiological normal state) and gas exchange of arterial blood, including normalization of important parameter of oxygen blood saturation, O₂Sat (up to 99-100% of normal value).

In the models of hypoxia of different etiologies (normobaric, hemic, tissue), the liposomal composition with cytochrome c provides increasing of life time of animals by 47-68%, that is, it has a universal anti-hypoxic activity. The composition has an antitoxic effect, reducing the duration of narcotic sleep by 33-40% in contrast to narcotic intoxication (Table 8).

By the level of confirmed pharmacological activity in experimental pathologies of different genesis, the liposomal composition produced by the claimed method is competitive both in respect of the product of prototype method (effectiveness of which in the models of different pathologies is less in 1.2-1.8 times) and with respect to analogue drugs (the effectiveness of which in models of different pathologies less in 1.3-2.4 times).

In general, the highest quality is inherent to the liposomal composition with cytochrome c, which is produced by the claimed method with accordance to the parameters set forth in Examples 1-8, and which are different from those provided for producing of the composition by the prototype method, namely: as lipids the following is used: phosphatidylcholine selected from (e)PC or (s)PC in a mixture with one or two lipids selected from the group of DPPG, DPPC, DSPC, DPG, PG, PI or DOTA at weight ratio of phosphatidylcholine:to other lipids of 0.3-2.0:1; dissolution of PC is carried out in ethyl alcohol and dissolution of other lipids—in chloroform with their following merge, wherein the volumetric ratio of ethyl alcohol:chloroform is 1:1.5-2.5; emulsification is performed at a weight ratio of 1:11.4-18.5 of cytochrome c:lipids respectively

adding a cryoprotectant solution, wherein a cryoprotectant is selected from lactose, trehalose, sucrose oligosaccharides, whereas the said solution contains 60-80% of total cryoprotectant; and homogenization with step-by-step increasing pressure from 300 to 800 atm monitoring the size of emulsion particles, after its completion a solution of selected cryoprotectant is added to emulsion, wherein solution contains 40-20% of total cryoprotectant, and the weight ratio of lipids:cryoprotectant is 1:5.5-7.2.

Deviating from the above parameters (Examples 9-12 and the prototype—Example 13), the implementation of the method is prolonged and somewhat complicated (Table 3—increasing of time for stirring during emulsification, cascade filtration and increasing of the filtration time after dispersing) and desired pharmaceutical quality of the targeted product as a liposomal composition with cytochrome c is not achieved (Table 4).

Accordingly, the high pharmaceutical quality of the targeted product determines for liposomal composition polyfunctional pharmacological efficacy and a higher level of anti-catalytic, anti-hypoxic and antitoxic activity and the ability to restore the hemostatic blood coagulation system for MBL, the composition contains cytochrome c, a mixture of lipids, and a cryoprotectant, providing its components defined by Examples 1-8, and other than those for a prototype-produced composition, namely: a mixture of lipids includes phosphatidylcholine selected from (e)PC or (s)PC in a mixture with one or two lipids selected from the group of DPPG, DPPC, DSPC, DPG, PG, PI or DOTA, a cryoprotectant is a oligosaccharide selected from lactose, trehalose or sucrose, wherein in the composition a weight ratio of cytochrome c:phosphatidylcholine:other lipids:cryoprotectant is 1:2.9-8.6:4.3-15.7:78.6-102.8, and a percentage ratio of cytochrome c:phosphatidylcholine:other lipids:cryoprotectant is (0.81-1.06)%:(3.03-8.26)%:(4.13-9.88)%:(78.67-83.30)%.

Variation of these parameters of composition (Examples 9-12 and prototype—Example 13) reduces the level of its pharmacological effects.

The achieved optimal combination of favorable production effectiveness and operations for implementation of a method with a reliable pharmaceutical identification and positive pharmacological properties of the targeted product demonstrates the advantages of the claimed method for the object of the invention, that is, obtaining of a liposomal drug containing cytochrome c. Produced liposomal composition with cytochrome c and with identified individual composition, for which the polyfunctional pharmacological effects and harmlessness were first established, advantageously differs from the prototype-produced liposomal product containing cytochrome c.

This justifies the reasonability of using the claimed method for obtaining a stable and high-quality liposomal drug containing cytochrome c and introducing of this liposomal composition with cytochrome c as a potential effective drug with a multifunctional pharmacotherapeutic effect for use in ophthalmology, cardiology and hematology.

TABLE 1
Parameters for implementing the claimed method for obtaining a
liposomal composition containing cytochrome c, and a prototype method
with respect to nature of lipids and cryoprotectants that are used thereof ¹⁾

| | Applied lipids | | | |
|---|---|---|---|---|
| | | Other lipids | | |
| Example | Phosphatidyl- | Lipid name | Weight ratio | Cryo- |
| No | choline | (conventional name) | of other lipids | protectant |

Proposed method

| | | | | |
|---|---|---|---|---|
| 1 | (e)PC | DPPG | — | Lactose |
| 2 | (s)PC | DPPG | — | Lactose |
| 3 | (e)PC | DPPG + DPPC | 1:1 | Trehalose |
| 4 | (s)PC | DSPC + DPG | 2.5:1 | Lactose |
| 5 | (e)PC | DPPG + PG | 1:1 | Trehalose |
| 6 | (e)PC | DPPG + PI | 2:1 | Sucrose |
| 7 | (e)PC | DPPG + DOTA | 2.5:1 | Lactose |
| 8 | (e)PC | DPPC + DOTA | 0.5:1 | Trehalose |
| 9 | (e)PC | DPPG + DSPC | 0.3:1 | Lactose |
| 10 | (e)PC | DPPC + DOTA | 0.5:1 | Trehalose |
| 11 | (s)PC | DSPC + DPG | 1:1 | Lactose |
| 12 | (e)PC | DPPG + PG | 2:1 | Lactose |

Prototype method

| | | | | |
|---|---|---|---|---|
| 14 | (e)PC ²⁾ | DPPG | — | Lactose |

¹⁾ in all Examples cytochrome c is an integral component of the process implementation.
²⁾ origin of the substance in the prototype is not identified.

TABLE 2
Parameters for implementing the claimed method for obtaining
a liposomal composition containing cytochrome c, and a
prototype method with respect to ratios of components of the
produced composition, which are used thereof

| | Weight ratio | | |
|---|---|---|---|
| Example | Cytochrome c: | | Lipids |
| No | lipids mixture * | other lipids:PC | mixture:cryoprotectant |

Claimed method

| | | | |
|---|---|---|---|
| 1 | 1:15.7 | 1:1.2 | 1:5.5 |
| 2 | 1:12.9 | 1:0.8 | 1:7.2 |
| 3 | 1:14.3 | 1:0.7 | 1:6.5 |
| 4 | 1:18.5 | 1:0.9 | 1:5.6 |
| 5 | 1:15.7 | 1:0.4 | 1:6.0 |
| 6 | 1:12.9 | 1:2.0 | 1:6.7 |
| 7 | 1:17.1 | 1:0.7 | 1:5.8 |
| 8 | 1:11.4 | 1:0.3 | 1:6.9 |
| 9 | 1:12.9 | 1:0.2 | 1:4.6 |
| 10 | 1:21.4 | 1:4.0 | 1:5.3 |
| 11 | 1:12.8 | 1:0.15 | 1:5.3 |
| 12 | 1:11.4 | 1:1.7 | 1:4.3 |

Prototype method

| | | | |
|---|---|---|---|
| 13 | 1:34.1 | 1:3.6 | 1:3.0 |

* a mixture of lipids is PC with other applied lipids indicated in Table 1.

TABLE 3
Parameters of the claimed method for obtaining a liposomal composition
containing cytochrome c, and a prototype method with respect to technical
data which are used thereof

| | Example No | | | | | | | | | | | | |
|---|---|---|---|---|---|---|---|---|---|---|---|---|---|
| | Claimed method | | | | | | | | | | | | 13- |
| | 1 | 2 | 3 | 4 | 5 | 6 | 7 | 8 | 9 | 10 | 11 | 12 | Prototype |
| A medium for | + | + | + | + | + | + | + | + | − | + | + | − | − |
| dissolution of | − | − | − | − | − | − | − | − | + | − | − | + | + |
| lipids: | | | | | | | | | | | | | |
| a) ethyl alcohol - | | | | | | | | | | | | | |
| PC, | | | | | | | | | | | | | |
| chloroform - | | | | | | | | | | | | | |
| other lipids, | | | | | | | | | | | | | |
| wherein ratio of | | | | | | | | | | | | | |
| ethyl alcohol:chloroform | | | | | | | | | | | | | |
| is 1: | | | | | | | | | | | | | |
| 1.5-2.5 (vol.) | | | | | | | | | | | | | |
| [symbol] ) all lipids: | | | | | | | | | | | | | |
| mixture of ethyl | | | | | | | | | | | | | |
| alcohol and | | | | | | | | | | | | | |
| chloroform (1:4) | | | | | | | | | | | | | |
| Parameters for | + | + | + | + | + | + | + | + | − | + | − | + | − |
| emulsification: | − | − | − | − | − | − | − | − | + | − | + | − | + |
| a) Medium: | 60 | 70 | 55 | 45 | 60 | 45 | 65 | 80 | 112 | 85 | 90 | 95 | 120 |
| mixture of | 80 | 69 | 62 | 65 | 80 | 70 | 60 | 60 | 0 | 40 | 50 | 20 | 0 |
| cytochrome c, | | | | | | | | | | | | | |
| cryoprotectant | | | | | | | | | | | | | |
| and buffered | | | | | | | | | | | | | |
| solution: | | | | | | | | | | | | | |
| mixture of | | | | | | | | | | | | | |
| cytochrome c and | | | | | | | | | | | | | |
| buffered | | | | | | | | | | | | | |
| solution: | | | | | | | | | | | | | |
| b) Stirring time, | | | | | | | | | | | | | |
| min | | | | | | | | | | | | | |
| c) amount of | | | | | | | | | | | | | |
| introduced | | | | | | | | | | | | | |
| cryoprotectant | | | | | | | | | | | | | |
| (% of total | | | | | | | | | | | | | |
| amount) | | | | | | | | | | | | | |
| Parameters | 300 | 300 | 300 | 300 | 300 | 300 | 300 | 300 | 300 | 300 | 200 | 300 | 900 |
| of | 600 | 600 | 600 | 500 | 600 | 700 | 500 | 600 | 400 | 500 | 600 | 500 | The |
| dispersion: | 800 | 800 | 800 | 800 | 800 | 800 | 800 | 800 | 800 | 900 | 800 | 500 | whole |
| a) Pressure | | | | | | | | | | | | | process |
| for cycles, | <150 | <145 | <150 | <145 | <150 | ≈145 | <150 | <150 | <160 | <130 | <130 | <150 | <145 |
| at: | | | | | | | | | | | | | |
| 1st | − | − | − | − | − | − | − | − | + | − | + | − | + |
| 2nd | + | + | + | + | + | + | + | + | − | + | − | + | − |
| 3rd | 20 | 31 | 38 | 35 | 20 | 40 | 30 | 40 | 100 | 60 | 50 | 80 | 100 |
| b) Particles | | | | | | | | | | | | | |
| size after | | | | | | | | | | | | | |
| dispersion, | | | | | | | | | | | | | |
| nm | | | | | | | | | | | | | |
| c) | | | | | | | | | | | | | |
| introduction | | | | | | | | | | | | | |
| of | | | | | | | | | | | | | |
| cryoprotectant | | | | | | | | | | | | | |
| solution: | | | | | | | | | | | | | |
| within | | | | | | | | | | | | | |
| dispersion | | | | | | | | | | | | | |
| process | | | | | | | | | | | | | |
| after | | | | | | | | | | | | | |
| dispersion | | | | | | | | | | | | | |
| c) amount | | | | | | | | | | | | | |
| of | | | | | | | | | | | | | |
| introduced | | | | | | | | | | | | | |
| cryoprotectant | | | | | | | | | | | | | |
| (% of | | | | | | | | | | | | | |
| total | | | | | | | | | | | | | |
| amount) | | | | | | | | | | | | | |
| Parameters | − | − | − | − | − | − | − | − | − | − | + | − | + |
| for filtration | + | + | + | + | + | + | + | + | + | + | − | + | − |
| after | 10 | 11 | 12 | 10 | 12 | 10 | 14 | 10 | 15 | 20 | 28 | 19 | 29 |
| dispersion: | | | | | | | | | | | | | |
| a) filtration: | | | | | | | | | | | | | |
| cascade of | | | | | | | | | | | | | |
| filters | | | | | | | | | | | | | |
| filter of | | | | | | | | | | | | | |
| 0.22 μm | | | | | | | | | | | | | |
| b) filtration | | | | | | | | | | | | | |
| average | | | | | | | | | | | | | |
| time (for 11). | | | | | | | | | | | | | |
| min | | | | | | | | | | | | | |

TABLE 4
Identification and pharmaceutical quality parameters of a liposomal composition obtained by
the claimed method and prototype method

| | Example No | | | | | | | | | | | | |
|---|---|---|---|---|---|---|---|---|---|---|---|---|---|
| | Claimed method | | | | | | | | | | | | 13- |
| | 1 | 2 | 3 | 4 | 5 | 6 | 7 | 8 | 9 | 10 | 11 | 12 | Prototype |
| Identification: | + | + | + | + | + | + | + | + | + | + | + | + | + |
| cytochrome c | + | + | + | + | + | + | + | + | + | + | + | + | + |
| PC | + | + | + | + | + | + | + | + | + | + | + | + | + |
| other | | | | | | | | | | | | | |
| lipids | | | | | | | | | | | | | |
| Encapsulation | 95.1 | 94.1 | 98.4 | 95.6 | 96.8 | 99.0 | 95.0 | 94.8 | 89.0 | 91.3 | 90.3 | 92.0 | 88.0 |
| level of | | | | | | | | | | | | | |
| cytochrome | | | | | | | | | | | | | |
| c into | | | | | | | | | | | | | |
| liposomes | | | | | | | | | | | | | |
| (% of | | | | | | | | | | | | | |
| introduced | | | | | | | | | | | | | |
| amount) | | | | | | | | | | | | | |
| Liposomes | 147/ | 150/ | 145/ | 145/ | 147/ | 147/ | 145/ | 150/ | 165/ | 150/ | 148/ | 165/ | 155/67 |
| size | 95 | 90 | 100 | 95 | 97 | 92 | 94 | 90 | 60 | 90 | 80 | 75 | |
| (nm)/% | 132/5 | 138/ | | 135/5 | 140/3 | 135/8 | 130/6 | 140/ | 130/ | 132/ | 125/ | 150/ | 62/33 |
| liposomes | | 10 | | | | | | 10 | 25 | 10 | 10 | 25 | |
| by the | | | | | | | | | 100/ | | 100/ | | |
| size: * | | | | | | | | | 15 | | 10 | | |
| a) after | 150/ | 150/ | 148/ | 148/ | 150/ | 150/ | 145/ | 152/ | 160/ | 145/ | 150/ | 160/ | 155/60 |
| freeze- | 95 | 90 | 100 | 95 | 95 | 92 | 95 | 90 | 55 | 90 | 75 | 80 | 60/25 |
| drying | 135/5 | 138/ | | 135/5 | 140/5 | 140/9 | 135/5 | 142/ | 130/ | 130/ | 130/ | 140/ | 52/15 |
| b) after 9 | | 10 | | | | | | 10 | 25 | 10 | 15 | 20 | |
| months | | | | | | | | | | 95/10 | | 100/ | |
| storage | | | | | | | | | | 65/5 | | 10 | |
| Oxidation | 0.24 | 0.28 | 0.22 | 0.20 | 0.24 | 0.25 | 0.26 | 0.22 | 0.33 | 0.24 | 0.31 | 0.30 | 0.31 |
| index, | | | | | | | | | | | | | *** |
| conv. unit | | | | | | | | | | | | | |
| Formation | 1.2/ | 1.5/ | 1.5/ | 1.0/ | 1.2/ | 1.2/ | 1.0/ | 1.0/ | 2.0/ | 1.0/ | 1.8/ | 2.0/ | 1.8/ |
| time/emulsion | 115 | 110 | 120 | 110 | 112 | 120 | 100 | 105 | 80 | 90 | 100 | 95 | 95 *** |
| stability | | | | | | | | | | | | | |
| (min)* | | | | | | | | | | | | | |
| pH of | 6.56 | 6.50 | 6.56 | 6.70 | 6.55 | 6.61 | 6.50 | 6.50 | 6.62 | 6.48 | 6.75 | 6.58 | 6.74 |
| emulsion* | | | | | | | | | | | | | *** |
| Emulsion | 320 | 330 | 310 | 330 | 320 | 340 | 350 | 330 | 380 | 340 | 360 | 330 | 380 |
| osmolality, | | | | | | | | | | | | | |
| mOsm/g**)*** | | | | | | | | | | | | | |
| Weight loss | 5 | 5 | 5 | 5 | 4 | 4 | 5 | 4 | 6 | 5 | 5 | 3 | 6 |
| within | | | | | | | | | | | | | |
| drying (%)*** | | | | | | | | | | | | | |
| Quantitative | 0.70 | 0.70 | 0.70 | 0.70 | 0.70 | 0.70 | 0.70 | 0.70 | 0.67 | 0.70 | 0.70 | 0.70 | 0.67 |
| content | 6.0 | 4.0 | 4.0 | 6.0 | 3.0 | 6.0 | 2.0 | 5.0 | 9.0 | 3.0 | 8.0 | 5.0 | 18.0 |
| (mg/ml)*: | 5.0 | 5.0 | 6.0 | 7.0 | 8.0 | 3.0 | 6.0 | 7.0 | 2.0 | 12.0 | 1.0 | 3.0 | 5.0 |
| cytochrome c | | | | | | | | | | | | | |
| PC | | | | | | | | | | | | | |
| other | | | | | | | | | | | | | |
| lipids | | | | | | | | | | | | | |

(+) - positive identification
*determined for an emulsion that is reconstructed by adding of 10 ml of the saline solution to the lyophilized product, the liposomal composition
**in accordance with the requirements of the State Pharmacopoeia of Ukraine (2.2.35), for ophthalmologic drugs the index of osmolality should be within the range of 300-380 mOsm/g
***not regulated by the prototype

TABLE 5
Content of liposomal composition with cytochrome c obtained by claimed
method and prototype method, by weight and percentage ratio of com-
ponents determined by established parameters of pharmaceutical quality

| | Weight ratio | | | | |
|---|---|---|---|---|---|
| Ex- | cytochrome c:PC: | Content (% wt) | | | |
| ample | :other lipids: | Cytochrome | | Other | Cryo- |
| No | :cryoprotectant * | c | PC | lipids | protectant |

Composition obtained by the claimed method

| | | | | | |
|---|---|---|---|---|---|
| 1 | 1:8.6:7.1:85.7 | 0.93 | 7.95 | 6.63 | 79.50 |
| 2 | 1:5.7:7.1:92.9 | 0.89 | 5.09 | 6.36 | 82.66 |
| 3 | 1:5.7:8.6:92.9 | 0.88 | 5.02 | 7.53 | 81.57 |
| 4 | 1:8.6:10.0:102.8 | 0.84 | 7.15 | 8.34 | 78.67 |
| 5 | 1:4.3:15.7:94.3 | 0.86 | 3.71 | 9.88 | 81.54 |
| 6 | 1:8.6:4.3:85.7 | 0.96 | 8.26 | 4.13 | 82.64 |
| 7 | 1:7.1:7.1:100.0 | 0.81 | 5.80 | 8.13 | 81.95 |
| 8 | 1:2.9:8.6:78.6 | 1.06 | 3.03 | 9.09 | 83.30 |
| 9 | 1:12.8:2.9:71.4 | 1.09 | 14.02 | 3.11 | 77.88 |
| 10 | 1:4.3:17.1:114.3 | 0.70 | 3.00 | 12.0 | 80.0 |
| 11 | 1:11.4:1.4:68.6 | 1.19 | 13.61 | 1.70 | 81.69 |
| 12 | 1:7.1:4.3:50 | 1.52 | 10.86 | 6.52 | 76.08 |
| 13 | 1:14.3:8.6:92.9 | 1.30 | 18.63 | 11.80 | 65.20 |

Composition obtained by the prototype method

| | | | | | |
|---|---|---|---|---|---|
| 14 | 1:26.7:7.4:103.7 | 0.72 | 19.22 | 5.33 | 74.72 |

* content of cryoprotectant (pharmaceutical excipient) is taken according to the introduced amount

TABLE 6
The efficacy of the claimed method and prototype method in respect of
the pharmacological activity of the liposomal composition with cytochrome
c obtained thereof for the effect on the state of cataract development, and
biochemical parameters of the eye lens in the model of irradiation cataract

| | | | | | Parameters of enzymes activity of and content | | | | |
|---|---|---|---|---|---|---|---|---|---|
| | | | | | of lipid peroxidation products in the lens¹⁾ | | | | |
| | The number of eyes with | | | | | | Catalase, | | |
| | cataract (%) at stage: | | | | MDA, | DK, | μkat/ | LDH, | AP, |
| | 1 | 2 | 3 | 4 | nmol/ml | nmol/ml | ml | μkat/ml | nkat/l |
| Parameter | 0 | 0 | 0 | 0 | 8.1 | 1.9 | 43.1 | 12.2 | 14.9 |
| initial level | | | | | | | | | |
| (normal) | | | | | | | | | |

Irradiation (26 weeks) without treatment (saline solution - 6 weeks)

| | | | | | | | | | |
|---|---|---|---|---|---|---|---|---|---|
| Negative | 0 | 0 | 50 | 50 | 13.8 | 3.2 | 26.0 | 16.2 | 25.1 |
| control | | | | | | | | | |

Irradiation (26 weeks) + introduction of the next products (6 weeks):
The proposed composition is obtained by the claimed method (examples)*⁾**

| | | | | | | | | | |
|---|---|---|---|---|---|---|---|---|---|
| 1 | 25.0 | 75.0 | 0 | 0 | 9.6 | 2.4 | 36.1 | 13.2 | 16.0 |
| 5 | 25.0 | 75.0 | 0 | 0 | 8.8 | 2.0 | 40.1 | 12.9 | 15.5 |
| 7 | 12.5 | 87.5 | 0 | 0 | 9.0 | 2.1 | 38.0 | 12.6 | 16.5 |
| 11 | 0 | 87.5 | 12.5 | 0 | 10.2 | 2.7 | 32.0 | 14.0 | 19.1 |

Composition obtained by the prototype method*⁾**

| | | | | | | | | | |
|---|---|---|---|---|---|---|---|---|---|
| 13 | 0 | 87.5 | 12.5 | 0 | 10.8 | 2.8 | 31.1 | 14.1 | 20.0 |

Prototype drug*

| | | | | | | | | | |
|---|---|---|---|---|---|---|---|---|---|
| Oftan | 0 | 87.5 | 12.5 | 0 | 11.5 | 2.7 | 29.9 | 14.6 | 21.8 |
| Katachrom | | | | | | | | | |

¹⁾MDA—malondialdehyde; DK—dien conjugates; LDH—lactate dehydrogenase; AP—acid phosphatase.
*p < 0.05 comparing to the parameter initial level
**p < 0.05 comparing to the control

TABLE 7
The efficacy of the claimed method and the prototype method for the
pharmacological activity of the liposomal composition with cytochrome
c obtained by these methods with respect to its effect on the hemostatic
blood coagulation system in acute massive blood loss (MBL)

| | | | | | Parameters of acid- | | | | |
|---|---|---|---|---|---|---|---|---|---|
| | Parameters of blood | | | | base homeostasis state and | | | | |
| | coagulation system¹⁾ | | | | gas exchange of blood²⁾ | | | | |
| | APTT | PT | TT | FG | pH | pCO₂ | HCO₃⁻ | O₂Sat | BB |
| Initial value | 39.4 | 41.0 | 47.2 | 2.07 | 7.465 | 38.62 | 22.52 | 99.29 | 46.9 |
| (before | | | | | | | | | |
| MBL) | | | | | | | | | |
| MBL | 29.0 | 28.1 | 43.2 | 1.67 | 7.222 | 34.64 | 17.37 | 90.40 | 38.8 |
| (30%)* | | | | | | | | | |

MBL + proposed composition obtained by the claimed method (examples)*⁾**⁾

| | | | | | | | | | |
|---|---|---|---|---|---|---|---|---|---|
| 1 | 33.7 | 39.6 | 48.0 | 1.99 | 7.442 | 36.64 | 24.21 | 99.34 | 49.34 |
| 3 | 34.6 | 39.2 | 47.0 | 1.98 | 7.415 | 36.85 | 24.0 | 99.20 | 47.95 |
| 4 | 33.8 | 40.0 | 48.0 | 2.00 | 7.450 | 37.44 | 23.65 | 99.0 | 46.02 |
| 8 | 37.9 | 40.5 | 48.2 | 2.02 | 7.460 | 37.70 | 24.10 | 99.61 | 48.30 |
| 9 | 32.5 | 36.5 | 46.3 | 1.89 | 7.341 | 36.0 | 21.75 | 97.20 | 45.26 |

MBL + composition according to the prototype method*⁾**⁾

| | | | | | | | | | |
|---|---|---|---|---|---|---|---|---|---|
| 14 | 32.4 | 36.6 | 46.8 | 1.90 | 7.345 | 36.0 | 21.80 | 96.40 | 44.49 |

MBL + analogue drug*

| | | | | | | | | | |
|---|---|---|---|---|---|---|---|---|---|
| Cytochrome c | 30.1 | 31.1 | 45.8 | 1.81 | 7.301 | 35.38 | 18.47 | 92.06 | 41.27 |

¹⁾APTT—activated partial thromboplastin time, s; PT—prothrombin time, s; TT—thrombin time, s; FG—fibrinogen, g/L.
²⁾pCO₂—partial pressure of CO₂, mmHg; O₂Sat—oxygen blood saturation, %; HCO₃⁻—plasma bicarbonate, mmol/L BB—total amount of anions in the blood, mmol/L.
*p < 0.05 comparing to the parameter initial value
*p < 0.05 comparing to the MBL group

TABLE 8
The efficacy of the claimed method and the prototype method for
the pharmacological activity of the liposomal composition with
cytochrome c obtained by these methods with respect to
hypoxia of different etiology and narcotic intoxication

| Parameters of pharmacological | | | |
|---|---|---|---|
| activity in pathology models: | | | |
| Lifetime at hypoxia, min/% * | | | Duration of sleep at |
| Normobaric | Gemic | Tissue | narcotic intoxication, |
| hypoxia | hypoxia | hypoxia | min/% * |

Composition obtained by the claimed method (Examples):

| | | | | |
|---|---|---|---|---|
| 1 | 119.8/48.5 | 16.9/53.6 | 15.2/60.0 | 45.0/38.6 |
| 2 | 119.0/47.5 | 16.7/51.8 | 15.1/58.9 | 44.8/38.9 |
| 3 | 122.1/51.3 | 17.3/57.2 | 15.8/66.3 | 44.0/40.0 |
| 4 | 119.0/47.5 | 16.6/50.9 | 15.2/60.0 | 44.5/39.3 |
| 5 | 122.4/51.7 | 17.0/54.5 | 15.7/65.3 | 44.1/39.8 |
| 6 | 123.0/52.4 | 17.4/58.2 | 16.0/68.4 | 44.0/40.0 |
| 7 | 119.1/47.6 | 16.8/52.7 | 15.7/65.3 | 44.8/38.9 |
| 8 | 119.6/48.2 | 16.7/51.8 | 15.8/66.3 | 44.1/39.8 |
| 9 | 108.1/33.9 | 15.5/40.9 | 13.9/46.3 | 49.0/33.1 |
| 10 | 109.3/35.4 | 16.0/45.5 | 14.2/49.5 | 48.9/33.3 |

Composition obtained by the prototype method

| | | | | |
|---|---|---|---|---|
| 13 | 106.5/31.9 | 15.3/40.1 | 13.9/46.3 | 55.3/24.5 |
| Cytochrome | 98.9/22.6 | 14.8/34.5 | 13.3/39.5 | 61.0/16.8 |
| c-analogue | | | | |
| Control- | 80.7/— | 11.0/— | 9.5/— | 73.3/— |
| saline | | | | |
| solution | | | | |

* min/%: min—absolute value of parameter in minutes; % parameter change as to control group in percent.
p < 0.05 comparing to the control

### INFORMATION SOURCES

1. Salem F. R.//Ann. Review of biochemistry.-1977.-V. 46.-P.299-329.
2. Drogovoz S. M. Pharmacology (Coursebook-guide), Kharkiv, 2007, P. 216-222.
3. Niesmann M. G.//Crit. Rev. Ther. Drug Carrier Syst..-2002.-N 9.-P.1-38.
4. Hossen M., Kajimoto K., Anita H. at al//Molecular Therapy.-2013.-V.21, N 3.-P. 533-541
5. Liposome technology & lipoome preparation/ed. G. Gregoriadis.-2007.-CRC Press, Informa Healthcare.-324 p.
6. Ebrahim Sh., Peyman G., Lee P.//Survey of ophtalmology.-2005.-V.50, N 2.-P.167-181
7. Zhang J., Guan P., Wang T. at al//J. Pharmacy and Pharmacology, 2009.-V.61, N 9.-P.1171-1178.
8. Nantes I., Kawai C., Pessoto K., Muqnol K.//Methods Mol. Biol. 2010.-V. 606, N 3.-P.147-165.
9. Zavada Z. H.//Acta Polon. Pharm & Drug Res., 2012.-V.69, N 1.-P. 107-111.
10. Patent of Ukraine No 44318 on utility model “Method of producing of liposomal cytochrome c”, A61K 9/00. Published on 25 Sep. 2009.
11. Patent of Russia No 2110990 on invention “Liposomal vesicle with cytochrome c”, A61K9/127. Publication date 20.05.1998.
12. Application No CN 101019836 on invention “Nanometer cytochrome liposome medicine and its preparation”, A61K38/06; A61K38/17; A61K47/24; A61K47/34; A61K47/42; A61K9/127; A61K9/19; A61P39/02; A61P43/00. Published on 22 Aug. 2007.
13. Application No EA201201592 on invention “Method of producing of liposomal form of cytochrome c”, A61K38/06; A61K38/17; A61K47/24; A61K47/34; A61K47/42; A61K9/127; A61K9/19; A61P39/02; A61P43/00, published on 30 Jun. 2014.
14. Lipids catalogue. Lipoid E PS S, Germany
15. Cytochrome c. Farmasino Pharm. (Jiangsu) Co., China
16. Cytochrome c. Calzyme Lab. Inc, USA
17. Metelicina E. P.//Problems of ecological and medical genetics and clinical immunology.-Kiev-Lugansk-Kharkiv: Planeta kopi.-2000.-Issuen. 3(29).-P. 221-233
18. Experimental study of harmlessness and pharmacological activity of eye medicines. Methodical recommendations of the SPC MOH of Ukraine//KHiB, 2003.-43 p.
19. Fulop A., Turoczi Z., Garbaisz D. et al//Europ. Surugical Res.-2013.-N 50.-P.57-70.
20. Levi M. Guidelines for diagnosis and management of disseminated intravascular coagulation//British J. of Hematology.-2009.-N 145.-P.23-33.
21. Guide for experimental (preclinical) study of new pharmacological substances//2nd ed. by R. U. Habrieva.-M.: Publishing house “Me [symbol] a”, 2005.-455 p.
22. European Convention for the Protection of Vertebrate Animals used for Research or Other Scientific Purposes of 18.03.1986: Verkhovna Rada of Ukraine, official web portal: International documentation (Council of Europe).

1. A method of producing of a pharmacologically active liposomal composition by forming a mixture of lipids solutions in organic solvents, drying it in a vacuum and emulsifying the composition in an aqueous medium containing cytochrome c, dispersing of an emulsion with the addition of a cryoprotectant, followed by filtration, sterile filtration and freeze-drying, wherein to the medium for emulsification a solution of cryoprotectant is introduced, the said solution contains 60-80% wt. of its total amount, and dispersion is carried out at step-by-step increasing pressure from 300 to 800 atm controlling the size of an emulsion particles; after dispersion, a solution of cryoprotectant is added, the said solution contains 40-20% wt. of total cryoprotectant amount.

2. The method of claim 1, wherein phosphatidylcholine selected from egg phosphatidylcholine or soybean phosphatidylcholine, with a mixture of one or two different lipids selected from the group consisting of dipalmitoylphosphatidylglycerol, dipalmitoylphosphatidylcholine, distearoylphosphatidylcholine, diphosphatidylglycerol, phosphatidylglycerol, phosphatidylinositol or dioleoyloxypropy trimethylammonium, is selected as lipids.

3. The method of claim 1, wherein in order to form a mixture of lipids solutions, phosphatidylcholine is dissolved in ethyl alcohol, and dissolution of other lipids are carried out in chloroform with following merge of the said solutions at volumetric ratio of ethyl alcohol:chloroform in a range of 1:1.5-1:2.5.

4. The method of claim 1, wherein a weight ratio of cytochrome c:lipids mixture in a range of 1:11.4-1:18.5 is used.

5. The method of claim 4, wherein for a mixture of lipids a weight ratio of other lipids:phosphatidylcholine in a range of 1:0.3-1:2.0 is used.

6. The method of claim 1, wherein oligosaccharide selected from the group consisting of lactose, trehalose or sucrose is used as cryoprotectant.

7. The method of claim 6, wherein a weight ratio of lipids mixture:cryoprotectant is in a range of 1:5.5-1:7.2.

8. Pharmacologically active liposomal composition containing cytochrome c, a mixture of lipids and a cryoprotectant, wherein a mixture of lipids comprising egg phosphatidylcholine or soybean phosphatidylcholine and at most two different lipids selected from the group consisting of dipalmitoylphosphatidylglycerol, dipalmitoylphosphatidylcholine, distearoylphosphatidylcholine, diphosphatidylglycerol, phosphatidylglycerol, phosphatidylinositol or dioleoyloxypropy trimethylammonium, whereas a weight ration of cytochrome c:phosphatidylcholine:other lipids:cryoprotectant is in a range of (0.81-1.06)%:(3.03-8.26)%:(4.13-9.88)%:(78.67-83.30)%.

9. The composition of claim 8, wherein the composition includes oligosaccharide as a cryoprotectant, which is selected from the group consisting of lactose, trehalose or sucrose.

10. The composition of claim 8 having anticataract effect.

11. The composition of claim 8 having ability to restore the hemostatic coagulation system in acute massive blood loss.

12. The composition of claim 8 having anti-hipoxia and antitoxic effect.
